(19) United States
(12) Patent Application Publication
(10) Pub. No.: US 2003/0172393 A1
(43) Pub. Date: Sep. 11, 2003

(54) Non-human transgenic mammal, method for producing same and its use

(76) Inventors: Holger Reichardt, Echterdingen (DE); Gunther Schutz, Heidelberg (DE); Klaus Kastner, Swartmore, PA (US); Peter Herrlich, Karlsruhe (DE)

Correspondence Address: Teresa Stanek Rea, BURNS, DOANE, SWECKER & MATHIS, L.L.P., P.O. Box 1404, Alexandria, VA, 22313-1404, US

(21) Appl. No.: 10/326,087

(22) Filed: Dec. 23, 2002

Related U.S. Application Data
(63) Continuation of application No. 09/623,991, filed on Feb. 6, 2001, now abandoned.
Application No. 09/623,991 is a 371 of international application No. PCT/DE99/00747, filed on Mar. 12, 1999.

(30) Foreign Application Priority Data
Mar. 12, 1998 (DE) 198 15 958.7

(51) Int. Cl. A01K 67/027; C12N 15/00

(52) U.S. Cl. 800/21; 800/3

(57) ABSTRACT

The present invention relates to a non-human mammal whose glucocorticoid receptor (GR) is modified as regards its inducing function. Furthermore, this invention concerns a process for the production of such a mammal as well as its use for testing chemicals, pharmaceutical preparations and therapeutic approaches.

The present invention relates to a non-human mammal which has a modified glucocorticoid receptor. Furthermore, the invention relates to a process for the preparation of such a mammal as well as its use for testing chemicals, pharmaceutical preparations and therapeutic approaches.

The glucocorticoid receptor (hereinafter referred to as GR) is a receptor present in many cells and activated by the binding of glucocorticoids. In this form, GR induces the expression of target genes. These are e.g. genes whose products play a part for the gluconeogenesis, the proliferation of erythrocyte precursor cells or the apoptosis of thymocytes. On the other hand, activated GR also represses the expression of target genes. These are e.g. genes which are activated by AP1 such as collagenase gene, or NFKB, such as TNFγ, II-2 or II-6.

It is considered to interfere selectively with the functions of GR. In particular, the repressing function appears to be of interest for this, since it is a possible starting point for interfering with the most differing diseases. Such diseases are e.g. disorders of the acute phase reaction, septic shock, asthma, acute respiratory distress syndrome, inflammatory diseases, autoimmune diseases such as rheumatoid arthritis or multiple sclerosis, neuropsychiatric diseases, diseases of the hypothalamic-pituitary adrenal axis, osteoporosis, diabetes, steroid-myopathy or skin diseases.

Therefore, it is the object of the present invention to provide a product by which it is possible to investigate selectively, and optionally interfere with, the repressing function of GR.

According to the invention this is achieved by the subject matters defined in the claims.

Thus, the subject matter of the present invention relates to a non-human mammal whose GR is modified as regards its inducing function. In particular, the inducing function is eliminated.

The present invention is based on the applicant's insight that the individual functions of GR can be modified separately from one another. He found that the inducing function of GR can be modified, in particular be eliminated, without the repressing function being affected by this. Furthermore, he found that the inducing function of GR is connected with its dimerization and DNA binding, respectively, and the inducing function of GR can be modified, in particular be eliminated, by mutation thereof, e.g. in the D loop of GR, in particular by point mutation A 458 T. the applicant also discovered that a GR modified as regards its inducing function still has immunosuppressive and antiinflammatory activity. The applicant obtained his insights from a transgenic mouse whose GR is modified as regards its inducing function (cf. Examples 2 and 3).

According to the invention the applicant's insights are utilized to provide a non-human mammal whose GR is modified, in particular eliminated, as regards its inducing function.

The expression “non-human mammal” comprises any mammal whose GR can be modified as regards its inducing function. Examples of such mammals are mice, rats, rabbits, horses, cattle, sheep, goats, monkeys or apes, pigs, dogs and cats, with mice being preferred.

The expression “GR whose inducing function is modified, in particular eliminated” comprises a GR of a non-human mammal whose repressing function is unchanged but which shows a modification of the inducing function. The, inducing function is preferably eliminated. This can be achieved e.g. by mutating the dimerization of GR and its DNA binding, respectively. This is obtained e.g. by a mutation in the D loop of GR, in particular by point mutation A 458 T.

A further subject matter of the present invention relates to cells obtained from the above non-human mammal. These cells may be present in any form, e.g. in a primary or long-term culture.

A non-human mammal according to the invention can be provided by common methods. It is favorable to employ a method which comprises the steps of:

(a) transfection of embryonal stem cells of a non-human mammal with a vector which enables recombination between the DNA, coding for the inducing function of GR, of the embryonal stem cells and a corresponding mutated DNA of the vector,

(b) isolation of cells stably transfected in (a) and introduction thereof into female animals of a non-human mammal, as well as

(c) analysis of the offspring obtained in (b) for a GR whose inducing function is modified, in particular eliminated.

The above explanations apply correspondingly to the expressions “non-human mammal” and “GR whose inducing function is modified, particularly eliminated”.

Furthermore, the expression “embryonal stem cells” relates to any embryonal stem cells of a non-human mammal suitable for mutation of the DNA coding for the inducing function of GR. The embryonal stem cells originate preferably from mice, in particular the cells E14/1.

The expression “vector” comprises any vector which by recombination with the DNA of embryonal stem cells enables a modification of the DNA coding for the inducing function of GR. The modification is preferably such that the inducing function of GR is eliminated. It is favorable for the vector to include a DNA which carries a mutation in a region which is necessary for the dimerization of GR and its DNA binding, respectively. In particular, the mutation may be within a region which codes for the D loop of GR. The mutation can be especially preferably the point mutation A 458 T. Moreover, it is favorable for the vector to include a marker by which a selection can be made as to present stem cells in which the desired recombination has been effected. Such a marker is e.g. the IoxP/tkneo cassette which can be removed from the genome again by means of the Cre/IoxP system. An above vector also relates to the subject matter of the present invention. Such a vector, i.e. the vector of Example 1 and FIG. 1, respectively, was deposited with the DSMZ [German-type collection of micro-organisms and cell cultures] as pmGR2-tr under DSM 12026 on Feb. 19, 1998.

In addition, the person skilled in the art knows conditions and materials to carry out steps (a)-(c). As regards the analysis made in (c), he will take into consideration e.g. of processes by which he can prove that the induction of genes is weakened or prevented whose products play a part in the gluconeogenesis, the proliferation of erythrocyte precursor cells or the apoptosis of thymocytes. Such processes are described below in the examples.

The present invention provides a non-human mammal whose GR is modified as regards its inducing function. This modification may be an elimination of the inducing function. The repressing function of GR can be investigated selectively with such a mammal and cells therefrom, respectively. Furthermore, it is possible by this to find substances, pharmaceutical preparations and therapeutic approaches by which a select influence can be exerted on the repressing function of GR. Therefore, the present invention provides a basis to exert an influence on the most varying diseases. Such diseases are e.g. disorders of the acute-phase reaction, septic shock, asthma, acute respiratory distress syndrome, inflammatory diseases, autoimmune diseases such as rheumatoid arthritis or multiple sclerosis, neuropsychiatric diseases, diseases of the hypothalamic-pituitary-adrenal axis, osteoporosis, diabetes, steroid-myopathy or skin diseases.

### BRIEF DESCRIPTION OF THE DRAWINGS

FIG. 1 shows the introduction of point mutation A 485 T into GR. (a) indicates the amino acid sequence of the second zinc finger in the DNA binding domain of GR. The exchange of alanine 458 by threonine is indicated. (b) mentions the strategy of recombination. The stars mark the position of point mutation. The modified locus represents the structure of the GR locus after the recombination, the end locus represents the structure after the additional recombination of the two IoxP sites (triangles) by Cre recombinase. (c) indicates the genotype by PCR. A 240 base pair fragment is amplified by using the primers indicated in (b) by arrows and cleaved by the restriction enzyme BsrG1 for which a new recognition site has been introduced by point mutation. The two 120 bp fragments which have been created by point mutation, are distinguished by the uncut fragment by gel electrophoresis. (d) indicates a sequence comparison. Brain RNA is amplified by RT-PCR, primers being used whose sequences are present in exons 3 and 5 of GR. The resulting fragments are subcloned and sequenced. The two mutated bases are indicated.

FIG. 2 shows the comparison between a mouse according to the invention and a wild-type mouse as regards the inducing function of GR. (a) indicates the induction of tyrosine aminotransferase (TAT). Dexamethasone is injected into a mouse according to the invention and into a wild-type mouse and liver-mRNA expression of TAT and β-actin, respectively, is analyzed as control by Northern blot. (b) states the induction of the proliferation of erythrocyte precursor cells. Erythrocyte precursor cells are isolated and cultured. Cumulative cell numbers are determined. (c) gives the induction of the apoptosis of thymocytes. The percentage of viable cells after the treatment with dexamethasone (hatched bars) or in controls (solid bars) is indicated.

FIG. 3 shows the induced inhibition of the AP-1-caused repression of the collagenase gene. Primary fibroblasts from a mouse according to the invention and a wild-type mouse, respectively, are treated with dexamethasone (Dex), TPA or both and mRNA amounts of MMP-13 (mouse collagenase 3) and GAPDH are analyzed as control by Northern blot.

FIG. 4 shows the induced expression of cytokine mRNA and its repression. Primary thymocytes from a mouse according to the invention and a wild-type mouse, respectively, are treated with ionomycin, phorbol ester (TPA), dexamethasone (dex) and a combination thereof (T+D), respectively. The mRNA expression of II-2 and TNFγ is determined by means of “RNase protection assay”. (A), (C) relate to the induced expression and repression of II-2 mRNA and its quantification. (B), (D) relate to the induced expression and repression of IFNγ mRNA and its quantification. (con) means control and (n, d) not detectable.

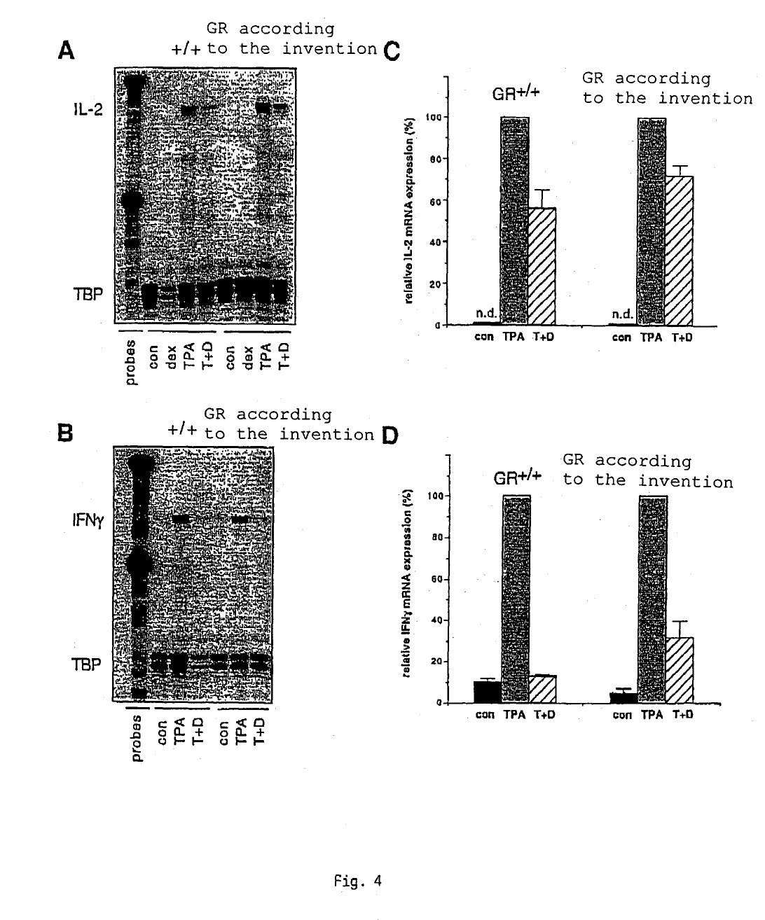

FIG. 5 shows the expression of cytokine mRNA. Macrophages from a mouse according to the invention and a wild-type mouse, respectively, are treated with lipopolysaccharide (LPS) and a combination of LPS and dex (L+D), respectively. The mRNA expression of TNF_{α} and II-6 is determined by means of “RNase protection assay”. (A), (B), (C) relate to the expression and repression of II-6 and/or TNF_{α} mRNA and its quantification. (con) means control.

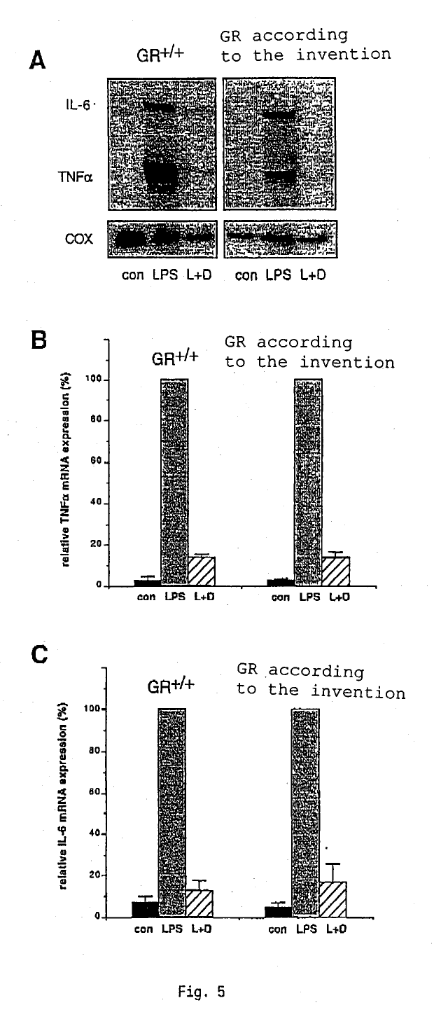

FIG. 6 shows the reaction of a mouse in the case of an infection and an inflammation, respectively. (A) A mouse according to the invention and a wild-type mouse, respectively, is treated with LPS, and the serum concentration of TNF_{α} is determined. (B) The skin of a mouse according to the invention and a wild-type mouse, respectively, is treated with TPA and a combination of TPA and dexamethasone (T+D), respectively, and the serum concentration of II-6 is determined. (C) The ear of a mouse according to the invention and a wild-type mouse, respectively, is treated with TPA and a combination of TPA and dexamethasone (T+D), respectively, and a certain region of the ear is cut out and its weight is determined as a measure of a resulting edema.

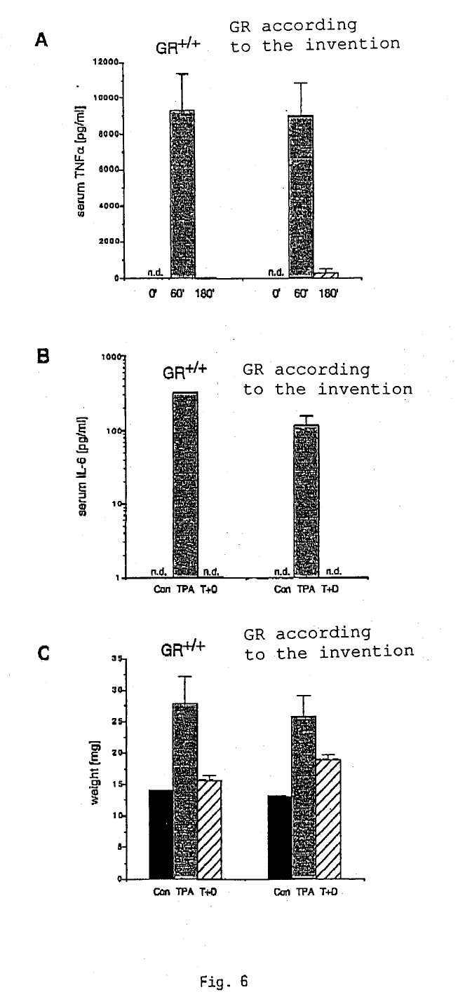

The invention is explained by the below examples.

### EXAMPLE 1

### Provision of a Non-Human Mammal According to the Invention

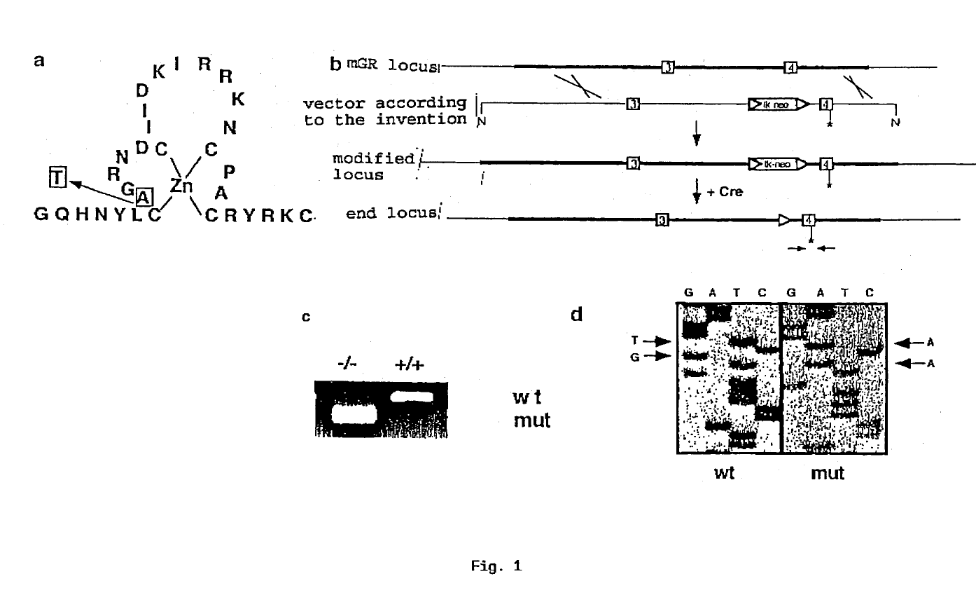

A non-human mammal is provided whose GR is eliminated as regards its inducing function. For this purpose, a vector is used which has about 11 kb of the GR gene, including exons 3 and 4. In addition, the vector shows point mutation in exon 4, so that the GR encoded by this has threonine in place of alanine at position 458. Moreover, the vector comprises an IoxP-tkneo selection cassette (cf. FIG. 1).

This vector is introduced by electroporation into the embryonal mouse stem cells E14/1. Stably transfected cells are obtained by selection with 350 μg/ml G418. These cells are subjected to a transient transfection with 20 μg of a Cre expression plasmid before 1 μM ganciclovir is added thereto. Thus, a selection is made for cells which have lost the selection cassette. These cells include the given point mutation in the GR gene and additionally about 50 base pairs along the remaining Iox P site in intron 3.

The latter cells are injected into mouse blastocysts which are then introduced into female mice. Chimeric mice are obtained from which heterocygous and homocygous mice are obtained by pairing.

### EXAMPLE 2

### Detection of Individual Functions of a Non-Human Mammal According to the Invention

The non-human mammal provided in Example 1 is employed. It is used in experiments which are specific to the individual functions of GR.

(a) Induction of Enzymes of Gluconeogenesis

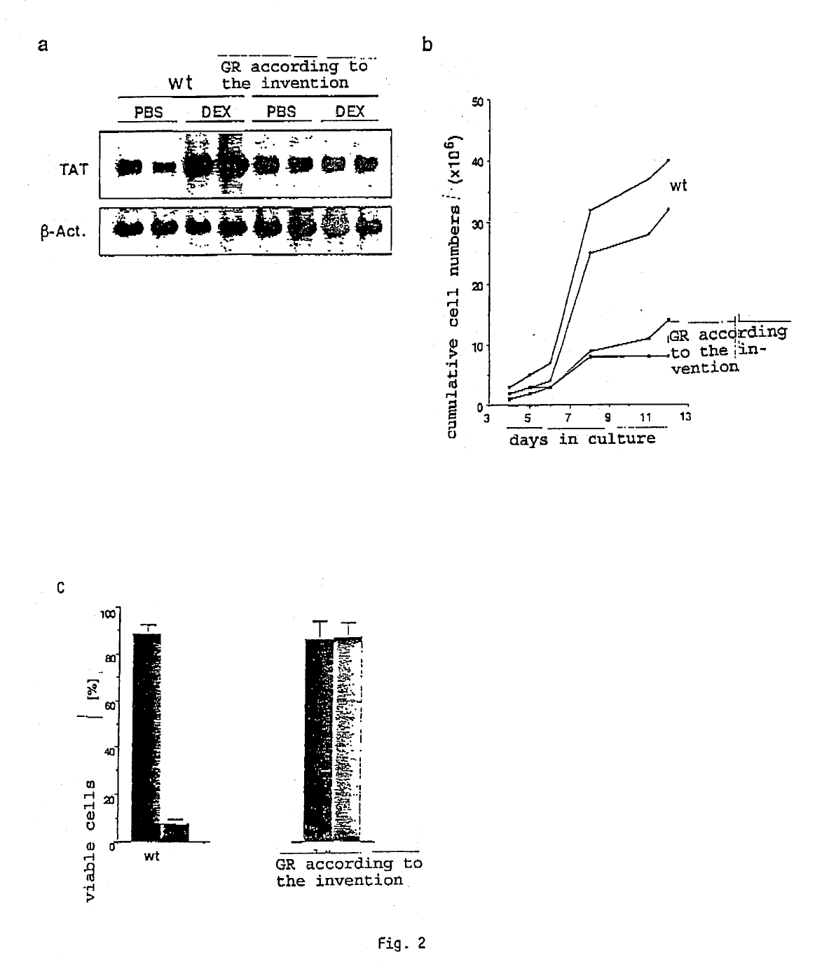

10 μg/100 g dexamethasone and PBS, respectively, are injected in each case into a mouse according to the invention and a wild-type mouse. The animals are killed after two hours. Liver RNA is isolated and the mRNA expression of tyrosine aminotransferase (TAT) and/or β-actin as control is determined in a Northern blot (cf. FIG. 2a).

It shows that a wild-type mouse but not a mouse according to the invention can induce TAT mRNA.

(b) Induction of the Proliferation of Erythrocyte Precursor Cells

Fetal liver is isolated each from a mouse embryo according to the invention and a wild-type mouse embryo and dispersed into individual cells. These cells are then proliferated in modified CFU-E medium, which contains SCF, hEpo, IGF-1 and dexamethasone. Growth kinetics is prepared daily by counting the cells using an electronic cell counter (cf. FIG. 2b).

It shows that a wild-type mouse but not a mouse according to the invention can induce the proliferation of erythrocyte precursor cells.

(c) Induction of the Apoptosis of Thymocytes

Thymocytes are isolated in each case from a 6-12-week-old mouse according to the invention and a correspondingly old wild-type mouse and cultured in RPMI medium for 24 hours in the presence and absence, respectively, of dexamethasone (10⁻⁶ M). The cells are then stained using propidium iodide and analyzed for their fluorescence intensity by means of FACS. The loss of DNA content is taken as a measure of apoptosis.

It shows that a wild-type mouse but not a mouse according to the invention can induce apoptosis of thymocytes.

(d) Inhibition of the AP-1-Caused Activation of the Collagenase Gene

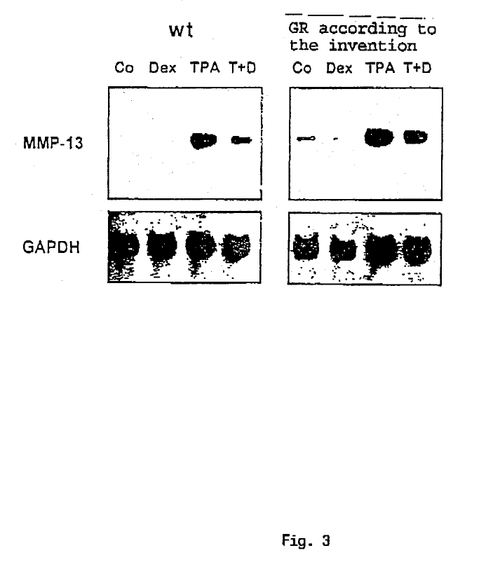

Primary fibroblasts are isolated from embryos of a mouse according to the invention and a wild-type mouse. For 6 hours, the fibroblasts are treated with 10⁻⁶ M dexamethasone, 10⁻⁷ M TPA or both before RNA is isolated and the mRNA expression of MMP-13 (mouse collagenase 3 gene) and of GAPDH as control are determined in a Northern blot (cf. FIG. 3).

It shows that a wild-type mouse and also a mouse according to the invention can inhibit the AP1-induced activation of the collagenase gene.

Thus, it becomes evident that a mammal according to the invention, e.g. a mouse, expresses a GR in which the repressing function is maintained but the inducing function is modified, in particular eliminated.

### EXAMPLE 3

### Investigations Made with a Non-Human Mammal According to the Invention and Cells Thereof, Respectively, as Regards Immunosuppressive Activity and Antiinflammatory Activity, Respectively

3.1 Investigations Made with Primary Thymocytes

Primary thymocytes from a mouse according to the invention and a wild-type mouse, respectively, are isolated and treated for 6 h with 0.5 μg/ml ionomycin as well as 10 μg/ml phorbol ester (TPA) and 10⁻⁶ M dexamethasone (dex) and a combination thereof (T+D), respectively. 2 μg of whole RNA are subjected in each case to an “RNase protection assay”.

It shows that a comparable expression of cytokin mRNA, e.g. II-2 and IFN_{γ} and a comparable repression, respectively, is obtained in primary thymocytes of both mice.

3.2. Investigations Made with Peritoneal Macrophages

Peritoneal macrophages from a mouse according to the invention and a wild-type mouse, respectively, are isolated 5 days after a thioglycolate treatment, and treated after 24 h for 2 h with 100 ng/ml lipopolysaccharide (LPS) and a combination of 10⁻⁶ M dexamethasone and lipopolysacahride (L+D), respectively. 0.5 μg of whole RNA is subjected in each case to an “RNase protection assay”.

It shows that a comparable expression of cytokine mRNA, e.g. TNF_{α} and II-6, and a comparable repression, respectively, is obtained in peritoneal macrophages of both mice.

3.3 Investigations Made in Connection with Mice

(A) 100 μg LPS each are administered to a mouse according to the invention and a wild-type mouse, respectively. The mice are killed at 0, 60 min. and 180 min. serum is isolated each and the TNF_{α} concentration is determined by a common ELISA kit.

It shows that the induction of TNF_{α} and its repression is comparable in both mice.

(B) The skin of the back of a mouse according to the invention and a wild-type mouse, respectively, is treated in each case with TPA and a combination of TPA and dexamethasone (T+D), respectively. Serum is isolated each and the II-6 concentration is determined by a common ELISA.

It shows that the induction of II-6 and its repression is comparable in both mice.

(C) The left ear of a mouse according to the invention and a wild-type mouse, respectively, is treated in each case with TPA and a combination of TPA and dexamethasone (T+D), respectively. A certain region of the ear is removed each, and the weight of the edema formed is determined.

It shows that the weight of the edema after the respective treatment is comparable in both mice.

Hence it is shown that a mouse according to the invention and its cells have immunosuppressive and antiinflammatory activity which can be compared with that of a wild-type mouse.

1. A non-human mammal whose glucocorticoid receptor (GR) is modified as regards its inducing function, the non-human mammal beingobtainable by a process, comprising the steps of:
(a) transfection of embryonal stem cells of a non-human mammal with a vector which enables a recombination between the DNA coding for the inducing function of GR, of the embryonal stem cells and a corresponding mutated DNA of the vector,
(b) isolation of cells stably transfected in (a) and introduction thereof into female animals of a non-human mammal, as well as
(c) analysis of the offspring obtained in (b) for a GR whose inducing function is modified.

2. The non-human mammal according to claim 1, wherein the inducing function is eliminated.

3. The non-human mammal according to claim 1 or 2, wherein GR is mutated as regards its dimerization and DNA binding, respectively.

4. The non-human mammal according to any one of claims 1 to 3, wherein GR has a threonine at position 458.

5. A process for the provision of a non-human mammal according to any one of claims 1 to 4, comprising the steps of:
(a) transfection of embryonal stem cells of a non-human mammal with a vector which enables a recombination between the DNA coding for the inducing function of GR, of the embryonal stem cells and a corresponding mutated DNA of the vector,
(b) isolation of cells stably transfected in (a) and introduction thereof into female animals of a non-human mammal, as well as
(c) analysis of the offspring obtained in (b) for a GR whose inducing function is modified.

6. The process according to claim 5, wherein the inducing function is eliminated.

7. The process according to claim 6, wherein GR is mutated as regards its dimerization and DNA binding, respectively.

8. The process according to any one of claims 5 to 7, wherein GR has a threonine at position 458.

9. Use of a non-human mammal according to any one of claims 1 to 4 for testing substances, pharmaceutical preparations and/or therapeutic approaches as regards the repressing function of GR.

10. Use according to claim 9, wherein the substances, pharmaceutical preparations and/or therapeutic approaches have to be understood as regards disorders of acute-phase reaction, a septic shock, asthma, an acute respiratory distress syndrome, inflammatory diseases, autoimmune diseases such as rheumatoid arthritis or multiple sclerosis, neuropsychiatric diseases, diseases of the hypothalamic-pituitary-adrenal axis, osteoporosis, diabetes, steroid myopathy or skin diseases.
